(19) United States
(12) Patent Application Publication
(10) Pub. No.: US 2007/0065524 A1
(43) Pub. Date: Mar. 22, 2007

(54) Pharmaceutical composition containing guaiacol derivatives and syringol derivatives extracted from natural plant vinegar

(75) Inventor: Sung-Ho Wang, Incheon (KR)

Correspondence Address: WELSH & KATZ, LTD, 120 S RIVERSIDE PLAZA, 22ND FLOOR, CHICAGO, IL, 60606, US

(73) Assignee: Oaky Natural Co., Ltd., Geumwang-eup (KR)

(21) Appl. No.: 10/578,583

(22) PCT Filed: Nov. 8, 2004
(86) PCT No.: PCT/KR04/02880

§ 371 Date: May 5, 2006

(30) Foreign Application Priority Data
Nov. 7, 2003 (KR) 10-2003-0078542
Feb. 19, 2004 (KR) 10-2004-0011194
Mar. 23, 2004 (KR) 10-2004-0019832
Apr. 28, 2004 (KR) 10-2004-0029745

(51) Int. Cl. A01N 31/14; A61K 31/075; A61K 36/82

(52) U.S. Cl. 424/725.1; 514/731; 514/718; 424/729

(57) ABSTRACT

The present invention relates to the pharmaceutical composition comprising mainly the guaiacol family compound and the syringol family compounds, extracted from natural plant vinegar. The present invention provides that pharmaceutical compositions has effects of treating oxidative toxicity, regulating blood glucose level, improving blood flow, treating hangover and treating atopic dermatitis as well as the safe composition to be free from acute toxicity, subacute toxicity etc . . . The pharmaceutical composition of the present invention can be used as an agent or an ingredient of health functional food.

### TECHNICAL FIELD

The present invention relates to a pharmaceutical composition including component extracted from natural plant vinegar as an effective ingredient.

### BACKGROUND ART

Timber becomes a charcoal smoking white if the timber is laid in a rare air place and an internal temperature of a burning furnace (a furnace burning charcoal) is 350-450° C. by heating. In the process, the crude natural plant vinegar, brown particle droplets are produced by dew condensation phenomena when smoke released from burning furnace is collected and is passed through cold smoke pipe.

The crude natural plant vinegar maintaining in the natural state for 6 months to 1 year is separated into the three of layers. The top layer is light oil, the middle layer is plant vinegar and the bottom layer is tar. pH 3 of Effective ordinary fundamental plant vinegar is obtained if only the middle later is isolated. These fundamental plant vinegar is known as including about 280 kinds of organic acid such as organic acid like formic acid, nitric acid, lactic acid, etc.; phenolic compounds such as phenol, cresol, 2,4- and 3,5-xylenol etc.; carbonyl compounds like formaldehyde, acetaldehyde, propionic aldehyde etc.; alcohol compounds like methanol, ethanol etc. and 13 kinds of uncommon elements such as mineral, germanium etc.

The advantage of previous nature plant vinegar was producing the major component ‘acetic acid’. After that, synthetic acetic acid of high purity was sold cheap and the use of the natural plant vinegar disappeared. The use of the natural plant vinegar was reopened before and after the World War II, but the utilization of the natural plant vinegar is not using components contained in the natural plant vinegar but using peculiar flavors and colors. For example, it was available to use the smoke flavors of the natural plant vinegar for the smoke effect in making ham, bacon, sausage etc. or to use food additives providing the colors of well done fish or meat etc. The utilization was simple.

In Japan, they tried to improve the symptoms of tinea pedis, atopic dermatitis, diabetes, hepatitis etc. by using the natural plant vinegar but the study was limited because safety on human body was not confirmed from harmful components (e.g. tar, methanol, benzopyren, mehtylcolarens) contained in the natural plant vinegar.

The natural plant vinegar is forest resource isolated from the smoke of a charcoal furnace. The natural plant vinegar is made by heat decomposition of components such as cellulose, lignin etc. contained in trees, and the natural plant vinegar includes greater than 200 kinds of organic components having various functions. However, safety on human body have been doubtful and could not have been regarded as healthful components because the natural plant vinegar included in bulk harmful components as well as effective components. In addition, functions of the natural plant vinegar could not have been confirmed in advance because effective constituents contained in the natural plant vinegar have not been identified and/or clearly classified.

Meanwhile, though oxygen free radical generated from body due to several causes has very short lifetime in the body compared with normal oxygen the free radical induces DNA damage and metabolism disorder by inactivating enzyme in the body and facilitates aging as well as cardiovascular disease like arteriosclerosis, musculoskeletal disease inducing biochemical aging of nervous tissue such as cataract or arthritis, or several kinds of malignant tumor and so on by greatly affecting cells and hormones. That is, the free radical is known as inducing brain disease of cerebrovascular accidents, levodopa etc., several kinds of disease of heart disease, ischemia, arteriosclerosis, dermatitis, digestive organ disease, inflammation, rheumatism, autoimmune disease and aging as well as cancer. Lipid peroxides produced from lipid components by the free radical peroxides lipid component destroys normal cells with other peroxides in the body. Thereby, several kinds of malfunction are brought about and also becomes a cause of diseases. (See B. Halliwell, Drugs 42:569, 1991; K. Fukuzawa and Y. Takaishi, J. Act. Oxyg. Free Rad. 1: 55, 1990; and J. Neuzil and J. M Gebicki, J. Med. 320: 915, 1989)

Therefore, antioxidant enzyme such as SOD (superoxide dismutase), GPO (glutathione peroxidase), catalase and antioxidant materials such as vitamin C and D protect the attack of free radical in the body in order to remove free radical generated from the body. However, cell balance between cell generation and cell death is destroyed and the aging of internal organs and tissue goes in progress because while grow older and older, an activity of antioxidant enzymes like SOD, GPO, catalase is declined and the content of antioxidant materials such as vitamin C and E is reduced, and so they can not prevent aggression of free radical in the body. Recently, it is also important to supply antioxidant components into the body from the outside because the balances of free radical's both formation and removal in vivo has been destroyed by environmental factors such as several kinds of pollution.

Synthetic antioxidant materials developed up to date are BHA (butylated hydroxy anisole), BHT (butylated hydroxy toluene), NDGA (nordihydro-guaiaretic acid) etc. and the natural antioxidant materials are antioxidant enzyme such as SOD, peroxidase, catalase, GPO etc. and non-enzymatic antioxidant materials of tocopherol (e.g. vitamin E), ascorbic acid (e.g. vitamin C), carotenoid, glutathione etc. However, synthetic antioxidant materials increase activity of hepatomegaly and the liver microsomal enzyme, and the part of antioxidant materials absorbed in the body can induce toxicity or allergy. And synthetic antioxidant materials have some defect that the materials are easily destroyed by heating due to be weak in heat. (Shahi, F. and Wanasundara, P., Phenolic antioxidant Critical Review in Food Science and Nutrition (1992)). Meanwhile, natural antioxidant has advantage that it is safe in the body in contrast with synthetic antioxidant, but has disadvantage that its effect is weak. Therefore, it has been keenly demanded the development of effective, new and safe in vivo natural antioxidant materials.

Diabetes is taken notice of high attack rate and serious acute, chronic complication and is roughly divided into insulin-dependant (Type I) and insulin-independent (Type II) clinically. Insulin-dependant diabetes is a sort of autoimmune disease induced by that insulin-secreting cell, β-cell, is destructed by means of that lymphocyte is infiltrated into the interior of pancreatic islet, and attacks at all ages. Insulin-independent diabetes means that insulin is secreted from β-cell but insulin in blood can't act by increase of resistance against insulin in a peripheral target organ. And Insulin-independent diabetes is occurred after 40 years old in human and is generally accompanied with adiposis.

A dietary treatment is kept pace with an exercise cure in insulin-independent diabetes and in the case not to be treated by these methods, oral hypoglycemic agent is used. The agent of Merformin or biguanide system applied generally to a pyknic patient and the agent of sulfonylurea system applied to a non-pyknic patient, as the oral hypoglycemic agent, is mainly used but each these agents are accompanied with side effect such as lactic acidosis and hypoglycemia. α-Glucosidase inhibitor like acarobose is used as hypoglycemic agent, which is developed newly in order to remove these side effects. This agent inhibits the function of α-glucosidase in the small intestine and so delays the absorption of glucose, and improves postcibal hyperglycemia and hyperinsulinemia, which are matters in a diabetes patient, and has an advantage of not inducing hypoglycemia at the same time. However, agents improving insulin resistance, which is the major problem of insulin-independent diabetes, have not been developed yet.

Thrombus, which is a lump of clotted blood in vessel, is known to affect cardiovascular system harmfully by preventing blood circulation. Hemocoagulation maintains normal hemostasis and protective function, thereby keeping the function of the body normally. But excessive activation of coagulation factors of blood plasma, platelet aggregation-facilitation, or erythrocyte deformability disorder destroy homeostasis of blood flow, thereby inducing cardiovascular system diseases such as arteriosclerosis and stroke which are circulation disorder. The homeostasis is maintained by balance between inhibitory reaction and activation reaction of hemostatic mechanism in normal vessel. But excessive hemostatic action and generation of blood coagulation induce disorder in blood flow and induce lesion such as thrombus by preventing the flow of blood.

The peoples are drinking a lot as one of the social activities. The main ingredient of liquor is alcohol, and alcohol taken in mainly converts into acetaldehyde through oxidation in the liver and a part of alcohol (about 10%) is eliminated as respiration, urine and sweat. Detoxification action of acetaldehyde is generally known to bring about hangover phenomenon such as headache, general malaise, fatigue, pot belly, emesis etc. after drinking. Concentration of acetaldehyde is high in a heavy hangover symptom compared with a light hangover symptom though the concentration of ethanol in blood is similar, so that it is shown that the major cause for hangover by alcohol is acetaldehyde. Atopic dermatitis is known well as a sort of allergy disease. Atopic dermatitis along with urticaria, allergic rhinitis, bronchial asthma etc. is one of representative diseases of allergy disease. The each finding about the pathologic examination of atopic dermatitis depends on steps of lesion, however if atopic dermatitis is at a chronic state, epidermis may be thick and be examined the infiltration of cells involved in several immune reactions. In particular, a langerhans cell importantly taking charge of primary defense of immune reaction is increased and has antigen-presenting ability increased abnormally. Mast cells are increased in number and shows up a state of degranulation. Serum IgE is increased in 80-90% of atopic dermatitis and serum IgE is in particular high when allergic rhinitis or asthma is accompanied.

There is a steroid agent as a curative medicine inhibiting the symptom of atopic dermatitis. The steroid agent, hormone secreted from adrenal gland is effective in inhibiting inflammation or allergy, in rheumatism and is often used as immunosuppressant inhibiting a rejection reaction of organ transplantation. But the steroid agent has serious problems such as side effects and dependency.

### DISCLOSURE OF INVENTION

The subject of the present invention provides the composition including a guaiacol compound and a syringol compound extracted from the natural plant vinegar, which is confirmed safety on human beings. Another subject of the present invention provides several uses of the natural plant vinegar.

In one embodiment, the present invention provides the composition including the guaiacol family compounds shown by following formula land the syringol family compounds shown by following formula 2, extracted from the natural plant vinegar.

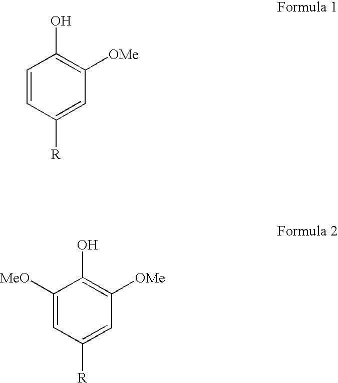

Where, in the formulas 1 and 2, R is hydrogen, alkyl, oxoalkyl or alkenyl.

In a preferred embodiment, the present invention provides a pharmaceutical composition for treating oxidative toxicity comprising the guaiacol family compounds of the formula 1 and the syringol family compounds the formula 2, extracted from the natural plant vinegar.

In another embodiment, the present invention provides a pharmaceutical composition for regulating blood glucose level comprising the guaiacol family compounds of the formula 1 and the syringol family compounds of the formula 2, extracted from the natural plant vinegar.

In certain embodiment, the present invention provides a pharmaceutical composition for improving blood flow comprising the guaiacol family compounds of the formula 1 and the syringol family compounds of the formula 2, extracted from the natural plant vinegar.

In another embodiment, the present invention provides a pharmaceutical composition for treating hangover comprising the guaiacol family. compounds of the formula 1 and the syringol family compounds of the formula 2, extracted from the natural plant vinegar and green tea leaves extract.

In another embodiment, the present invention provides a pharmaceutical composition for improving blood flow comprising the guaiacol family compounds of the formula 1 and the syringol family compounds of the formula 2, extracted from the natural plant vinegar and green tea leaves extract.

In another embodiment, the present invention provides a pharmaceutical composition for treating hangover comprising the guaiacol family compounds of the formula 1 and the syringol family compounds of the formula 2, extracted from the natural plant vinegar and green tea leaves extract.

In another embodiment, the present invention provides a pharmaceutical composition for treating atopic dermatitis comprising the guaiacol family compounds of the formula 1 and the syringol family compounds of the formula 2, extracted from the natural plant vinegar.

In another embodiment, the present invention provides a pharmaceutical composition for treating atopic dermatitis including the guaiacol family compounds of the formula 1 and the syringol family compounds of the formula 2, extracted from the natural plant vinegar and herbal extract with anti-allergic effect.

As stated above, the natural plant vinegar includes more than 200 kinds of organic ingredients estimated to have several effects, but also includes a lot harmful constituents such as tar, methanol, benzopyren. Moreover, a pharmaceutical effect of the natural plant vinegar has not been characterized because effective constituents included in the natural plant vinegar have not been classified and identified. Therefore, the present inventor developed a method to remove the harmful constituents from the natural plant vinegar (see Korea issued patent NO. 0290986 and 0212472), and classified and identified effective constituents of the natural plant vinegar purified by the method. The present inventor studied a lot in order to characterize a pharmaceutical and physiological effect. In the result, the composition of the present invention including the guaiacol compounds and the syringol compounds, extracted from the natural plant vinegar have a antioxidant function, a blood glucose level control function, an improvement of blood flow, an treatment of hangover, a treatment of atopic dermatitis, so the present inventor invented that the composition of the present invention can be used as those functions.

Besides, the present inventor experimented on several kinds of safety test of the composition of the present invention based on that the prior natural plant vinegar couldn't be used due to several side effects, and so the present inventor and confirmed that the composition of the present invention comprising the guaiacol compounds and the syringol compounds are safe on human beings.

### BRIEF DESCRIPTION OF THE DRAWINGS

FIG. 1 shows an example of the guaiacol compounds extracted from the natural plant vinegar.

FIG. 2 shows an example of the syringol compounds extracted from the natural plant vinegar.

FIG. 3a shows an example of a result about a pharmaceutical composition of the present invention comprising the guaiacol compounds and the syringol compounds in platelet aggregation assay using thrombin.

FIG. 3b shows an example of a result about a pharmaceutical composition of the present invention comprising the guaiacol compounds and the syringol compounds in platelet aggregation assay using collagen.

FIG. 4a shows an example of a result about the pharmaceutical composition of the present invention comprising the guaiacol compounds, the syringol compounds and green tea leaves extract in platelet aggregation assay using thrombin.

FIG. 4b shows an example of a result about the pharmaceutical composition of the present invention comprising the guaiacol compounds and the syringol compounds in platelet aggregation assay using collagen.

FIG. 5a is a microscopic picture of a negative control group administrating excipient in effect evaluation assay using NC/Nga mouse.

FIG. 5b is a microscopic picture of a positive control group administrating tacrolimus ointment in effect evaluation assay using NC/Nga mouse.

FIG. 5c is a microscopic picture of a test group administrating the compound of the present invention in effect evaluation assay using NC/Nga mouse.

### BEST MODES FOR CARRYING OUT THE INVENTION

The present invention provides a functional composition including the guaiacol family compounds shown by the following formula 1 and the syringol family compounds shown by the following formula 2, extracted from the natural plant vinegar.

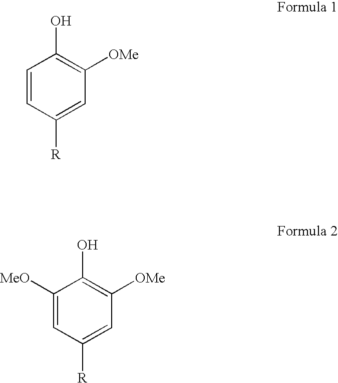

Where, in the formulas 1 and 2, R is hydrogen, alkyl, oxoalkyl or alkenyl.

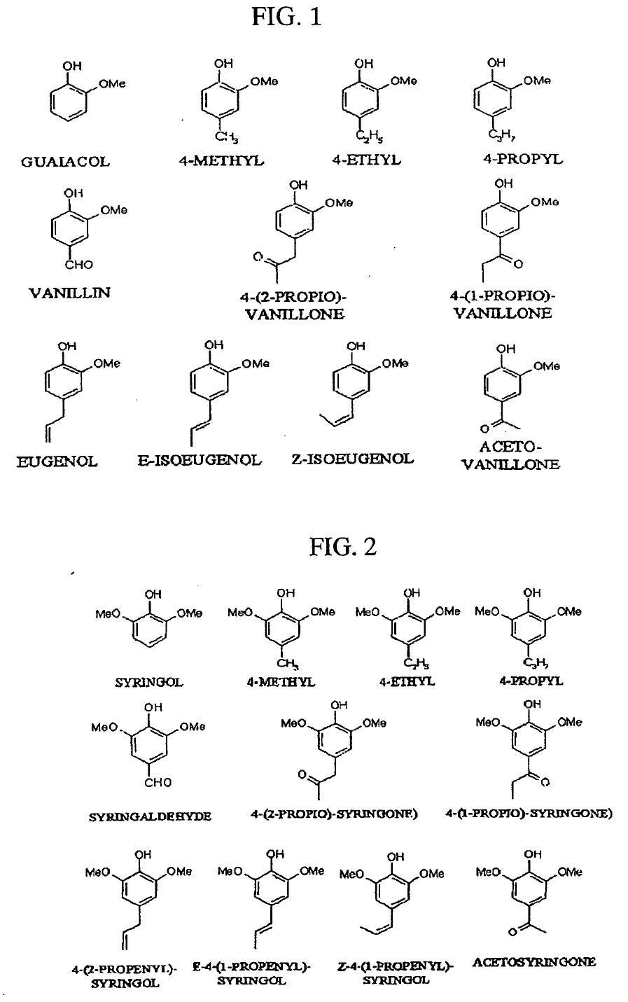

For examples of the guaiacol compound, there are guaiacol, 4-methylguaiacol, 4-ethylguaiacol, 4-propylguaiacol, vanillin, 4-(2-propio)-vanillone, 4-(1-propio)-vanillone, eugenol, E-isoeugenol, Z-isoeugenol, acetovanillon etc. FIG. 1 shows all of the compounds.

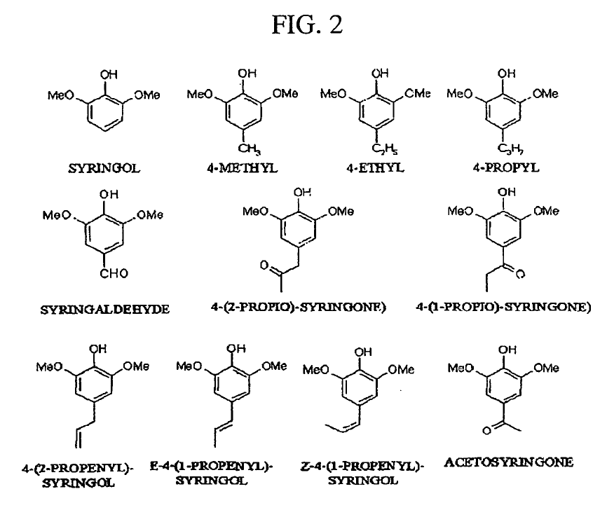

Examples of the syringol compound, syringol, 4-methylsyringol, 4-ethylsyringol, 4-propylsyringol, syringaldehyde, 4-(2-propio)-syringone, 4-(1-propio)-syringone, 4-(2-propenyl)-syringol, E-4-(1-prophenyl)-syringol, 7-(1-prophenyl)-syringol, acetosyringone etc. FIG. 2 is representative of the compounds.

Preferably, the pharmaceutical composition of the present invention including the guaiacol family compounds and the syringol family compounds, extracted from the natural plant vinegar contains 10⁻⁶ to 90 weight % of the guaiacol compound and 10⁻⁶ to 90 weight % of the syringol compound by the total weight of the compound.

Generally, it is impossible to heat pharmaceutical effective constituents during manufacture procedure because most of the pharmaceutical effective constituents commonly used are unstable to heat. On the while, the guaiacol family compounds and the syringol family compounds are compounds extracted from the natural plant vinegar and the natural plant vinegar used to extract has advantage of being very stable to heat due to compounds obtained from heat decomposition of trees.

Identifying and separating effective constituents from the purified natural plant vinegar can be performed by a method commonly used in pharmaceutical field. For example, there are separation method using column and extraction method using an organic solvent etc. More particularly isolating effective constituents from the purified natural plant vinegar can be separated by using an organic solvent like ether. In preferable embodiment, effective constituents from the purified natural plant vinegar can be obtained by acid and alkali treatment of extract obtained using an organic solvent like ether. Effective constituents from the purified natural plant vinegar can be performed by using GC-MSD.

The phenolic fraction among acid fraction, phenolic fraction, neutral fraction and basic fraction from the purified natural plant vinegar has several preferable effects. Useful effective constituents of the phenolic fraction are the guaiacol family compounds and the syringol family compounds, and these may have several preferable effects as effective constituents of phenolic fraction. The guaiacol and syringol compounds are highly volatile polyphenolic compounds as constituents with peculiar smell of the purified natural plant vinegar.

In preferable embodiment, the present invention provides the pharmaceutical composition for treating oxidative toxicity comprising the guaiacol family compounds of the formula 1 and the syringol family compounds of the formula 2, extracted from the natural plant vinegar.

The antioxidant effect of the pharmaceutical composition of the present invention comprising the guaiacol family compounds and the syringol family compounds, extracted from the natural plant vinegar, can be assayed by several common methods. For example, a method using DPPH(1,1-diphenyl-2-picrylhydrazin) can be used. DPPH, the free radical, in vitro used widely to screen the antioxidant effect of a natural substance. As concentration the pharmaceutical composition for treating oxidative toxicity is higher, removal efficiency of free radical is increased. In this method, liquid preparation including the 10 weight % of the pharmaceutical composition of the present invention can remove approximately 93% of DPPH and liquid preparation including 5 weight % of it can remove approximately 89% of DPPH and liquid preparation including 1 weight % of it can remove approximately 60% of DPPH. This means that the pharmaceutical composition of the present invention including the guaiacol compounds and the syringol compounds has excellent antioxidant activity.

Another method to assay the antioxidant effect is antioxidant enzyme assay using a mouse. For example, after administering the pharmaceutical composition of the present invention to the mouse during 2 weeks, bromobenzene(BB) is injected peritoneally to the mouse at intervals of 12 hours for 2 days. After 24 hours in BB injection, activity of the antioxidant enzyme is measured by sacrificing the mouse. In the method, a liquid preparation including 1 weight % of the pharmaceutical composition of the present invention can increase an antioxidant enzyme activity of Glutathion-s-transferase and Epoxide hydroxylase about 8.4% and 125% respectively and can decrease harmful materials, malondialdehyde(MDA), formaldehyde(AD) and p-aminophnol(AH), about 37%, 19% and 34% respectively.

The pharmaceutical composition of the present invention to treat oxidative toxicity includes the guaiacol compounds and the syringol compounds having a good antioxidant effect as effective constituents, so that it can be useful for brain related diseases like stroke and parkinson's disease, heart disease, ischemia, arteriosclerosis, dermatological disorder, digestive disorder, inflammation, rheumatism, autoimmune disease and aging etc.

In preferable embodiment, the present invention provides the pharmaceutical composition for regulating blood glucose level comprising the guaiacol family compounds of the formula 1 and the syringol family compounds of the formula 2, extracted from the natural plant vinegar.

The blood glucose level control effect of the pharmaceutical composition comprising the guaiacol compounds and the syringol compounds, extracted from the natural plant vinegar, can be assayed by using several common methods. For example, a diabetic animal model, db/db mouse, can be employed. The blood glucose level control effect of the pharmaceutical composition can be assayed through improvement degree of blood glucose concentration in Type II a diabetic animal, db/db mouse, when the composition of the present invention is orally administrated during 6 weeks. In more detail, the blood glucose control effect of the pharmaceutical composition comprising the guaiacol compounds and the syringol compounds, extracted from the natural plant vinegar, can be assayed by analyzing neutral fat, total cholesterol, glycosylated hemoglobin in blood. The composition of the present invention has excellent blood glucose level control ability in all of the aforementioned estimations in compared with a control group.

In another preferable embodiment, the present invention provides the pharmaceutical composition for improving blood flow comprising the guaiacol family compounds of the formula 1 and the syringol family compounds of the formula 2, extracted from the natural plant vinegar. More preferably, the pharmaceutical composition for improving blood flow further comprises green tea leaves extract. Epicatechin(EC), epicatechin gallate(ECG), epigallocatechin(EGC), epigallocatechin gallate(EGCG) are included in green tea leaves-extract and these polyphones are known to have several effects such as inhibitory activity against oxygen free radical, mutagenesis of carcinogen, and reuptake of cholesterol; and antibacterial and antiviral effects etc.

The present invention provides an surprising fact, which improving blood flow is increased synergistically in the case of using green tea leaves extracts with these pharmaceutical effects in conjugation with the guaiacol compounds and the syringol compounds, extracted from the natural plant vinegar.

Preferably, the composition of the present invention comprising the guaiacol compounds and the syringol compounds, extracted from the natural plant vinegar, and green tea leaves extracts includes 10⁻⁶ to 95 weight % of both the guaiacol compound and the syringol compound and 0.01 to 30 weight % of the green tea leaves extract on the basis of the total weight of the composition. More preferably, the composition of the present invention includes 10⁻⁶ to 90 weight % of the guaiacol compound and 10⁻⁶ to 90 weight % of the syringol compound and 0.01 to 30 weight % of the green tea leaves extract. The composition of the present invention has the most preferable improvement effect in the above mentioned amounts.

The improvement effect in blood flow of the pharmaceutical composition of the present invention can be assayed by using common methods in the art. For example, this assay may be performed based on the effect on platelet aggregation induced by thrombin, collagen etc. or based on the effect on serotonin secretion. In endogenous blood coagulation pathway, if materials like the collagen destroy the platelet by combining with blood coagulation factors in blood, thrombokinase in the platelet is released, and cooperative action of thrombokinase and calcium ion changes prothrombin into thrombin. The thrombin changes fibrinogen into fibrin, and the fibrin produces a blood clot in combination with blood cells in blood. That is, effects on improvement in blood flow can be known if the platelet aggregation is assayed by adding thrombin and collagen to blood. In addition, serotonin is stored in small vesicles of platelet and then, if platelet is activated by a stimulus of thrombin, serotonin is secreted out of platelet by the fusion of small vesicles and cell membrane, so that the activation of platelet and vasoconstriction are induced. Thereby, the effect of improvement in blood flow of the composition of the present invention can be assayed by measuring serotonin release. By these two methods, it can be confirmed that the pharmaceutical composition of the present invention comprising the guaiacol compounds and the syringol compounds, and the pharmaceutical composition of the present invention comprising the guaiacol compounds and the syringol compounds, extracted from the natural plant vinegar, and green tea leaves extract have good effects on improvement in blood flow.

In other preferable embodiment, the present invention provides the pharmaceutical composition for treating hangover comprising the guaiacol family compounds of formula 1 and the syringol family compounds of formula 2, extracted from the natural plant vinegar. More preferably, the composition for treating hangover further comprises additionally green tea leaves extract. The present invention provides also a surprising fact, which treatment effect on hangover is increased synergistically in the case of using green tea leaves extracts with these treatment effects in conjugation with the guaiacol compounds and the syringol compounds, extracted from the natural plant vinegar.

Preferably, the composition of the present invention comprising the guaiacol compounds and the syringol compounds, extracted from the natural plant vinegar, and green tea leaves extracts includes 10⁻⁶ to 95 weight % of the guaiacol compounds and the syringol compounds together and 0.01 to 30 weight % of the green tea leaves extract on the basis of the total weight of the composition. More preferably, the composition of the present invention includes 10⁻⁶ to 90 weight % of the guaiacol compounds and 10⁻⁶ to 90 weight % of the syringol compounds and 0.01 to 30 weight % of the green tea leaves extract. The composition of the present invention has the most preferable treatment effect on hangover in the above mentioned amounts.

The treatment effect on hangover of the composition of the present invention can be assayed by using common methods used in the art. For example, this assay can be conducted by measuring the concentration of acetaldhyde, which is estimated as a cause substance of hangover. By the method, it can be confirmed that the composition of the present invention comprising the guaiacol compounds and the syringol compounds, and the composition of the present invention comprising the guaiacol compounds and the syringol compounds, extracted from the natural plant vinegar, and green tea leaves extract have good effects on treating hangover.

In other preferable embodiment, the present invention provides the pharmaceutical composition for atopic dermatitis comprising the guaiacol family compounds of formula 1 and the syringol family compounds of formula 2, extracted from the natural plant vinegar. More preferably, the composition for the treatment of atopic dermatitis further comprises herbal extract with an anti-allergic effect.

Preferably, the composition of the present invention comprising the guaiacol compounds and the syringol compounds, extracted from the natural plant vinegar comprises respectively 10⁻⁶ to 90 weight % of the guaiacol compounds and the syringol compounds and 0.05 to 50 weight % of the herbal extract with an anti-allergic effect on the basis of the total weight of the composition. More preferably, the composition of the present invention comprises 10⁻⁶ to 30 weight % of the guaiacol compounds and 10⁻⁶ to 40 weight % of the syringol compounds and 0.05 to 50 weight % of the herbal extract with the anti-allergic effect. The composition of the present invention has the most preferable atopic dermatitis effect in the above mentioned amounts.

A treatment effect of herbal extracts, which has been commonly used up to date, was not enough to treat atopic dermatitis. The present invention provides also a surprising fact, which the atopic dermatitis effect is increased synergistically in the case that herbal extracts with a deficient curative effect mix with the guaiacol compounds and the syringol compounds, extracted from the natural plant vinegar. There are but not limited to Korean angelica (Angelicagigas) extract inhibiting release of histamine, Cnidium Rhizome (Cnidium officinale Makino) extract inhibiting pruritus etc. as plant extracts having the antiallergic effect. Plant extracts with the anti-allergic effect can be used, e.g. paeonia japonica extract, liquordice root (Glicyrrhizae Radix) extract, hoelen (Poria cocos Wolf) extract, scutellaria root (Scutellaria baicalensis Georgi) extract, schizandra fruit (Schizandra chinensis Baillon) extract, ginger extract, paconia japonica extract, rehmanniae radix preparata extract, salviae miltiorrhizae root (Salvia Miltirrhiza Bunge) extract, atractylodes rhizome white (Atractylodes japonica Koidzumi) extract, lycium fruit (Lycium Chinense Miller) extract, dried mushroom (Ganoderma lucidum Karsten) extract, cymanchum wilfordii extract, ginseng extract.

The atopic dermatitis effect of the composition of the present invention can be assayed by using an animal model commonly used in the art. For example, NC/Nga mouse can be employed. More particularly, the effect of the present invention can be assayed based on the decreasing degree of lesion of the atopic animal model, NC/Nga mouse, in case of orally administrating the composition of the present invention during 4 weeks. The composition of the present invention has better improvement effect than the existing medical supplies used widely for the treatment of atopic dermatitis when judged based on gross finding and pathologic finding of skin.

Until now, the natural plant vinegar could not be used due to uncertain safety. Therefore, the safety of the composition of the present invention comprising the guaiacol compounds and the syringol compounds, extracted from the natural plant vinegar, becomes a very important factor. The safety can be confirmed through common safety test in pharmacy art. For example, human safety test of the composition of the present invention can be performed by the toxicity assays such as acute toxicity test, genetic toxicity test and subacute toxicity test etc.

In the acute toxicity test, 5000 mg/kg, 50 times of daily uptake content (100 mg/body weight kg) of the composition in present invention is employed and the experiment is performed dividing into each six group (5 per each group) with 5 test group having the constant amount and 1 control group. Mortality, clinical manifestation, weight change, and anatomic pathologic finding were evaluated. These were examined every hour for 6 hours after administration on the day administrating the composition and were evaluated by examining general condition change, intoxication symptom and death or not of the animals once a day from the next day to 14^{th} days. The composition of the present invention was evaluated as the very safe compound through the acute toxicity test. As a result of the acute toxicity test, the pharmaceutical composition of the present invention comprising the guaiacol compounds and the syringol compounds, extracted from the natural plant vinegar, doesn't show any acute toxicity when being orally administrated to the mouse and the value of LD50 is estimated more than 5000 mg/weight kg, and the dose is confirmed as a safe dose when 50 times the quantity of the dose is orally administrated.

The genetic toxicity test was evaluated by using reverse mutation test using Salmontella typhimurium, chromosome abnormality test using a cultured mammalian cell and micronucleus test using rodent marrow cell. The composition of the present invention doesn't induce the reverse mutation in the range of the test application concentration 62-5000 ug/plate of the reverse mutation test using S. typhimurium TA1535, TA1537, TA98, and TA100. And the composition of the present invention doesn't induce the chromosome abnormality in the rang of the test application concentration 1.25-5 mg/ml of the chromosome abnormality test using a cultured mammalian cell. The composition of the present invention also doesn't induce the micronucleus in the range of the test application concentration 1250-5000 mg/kg of the micronucleus test. The results mean that the composition of the present invention is very safe substance not to show the genetic toxicity.

The subacute toxicity test was measured in the examination of death animals, general symptoms and weight changes during a period of administration, after orally administrating the pharmaceutical composition of the present invention in the amount of 0.5, 1.0, 2.5, 5.0 g/kg/day to ICR female and male mice 6 times a week for total 28 days. After administrating finally, gross autopsy finding, organ weight measurement, hematological and blood biochemical test, and histopathological test were conducted. The composition of the present invention is judged to be safe in above all of the evaluation items and is confirmed that non-toxic amount of the composition of the present invention is more than 5.0 g/kg/day.

The composition of the present invention including the guaiacol compounds and the syringol compounds, extracted from the natural plant vinegar, can further comprise excipients, disintegrants, binder, lubricant, sweeteners, coloring agent, flavor commonly used in the art and the composition can be formulated to tablets, capsules, powders, granules, suspensions, emulsions, syrups, liquids and solutions, unit dosage form like parentaral administrating preparation or pharmaceutical preparation for several administration. The composition of the present invention comprising the guaiacol compounds and the syringol compounds, extracted from the natural plant vinegar, can be administrated orally according to need and the composition of the present invention, as a effective components for daily, can be administrated 0.001 to 0.5 g/kg weight, preferably 0.01 g to 0.2 g, as a single-dose and a divided dose. A dose level about specific entity depends on weight, age, sex, a healthy state, a diet, administrated time, an administrated method, an excretion rate and a degree of diseases.

The present invention is explained concretely through embodiments. However, these embodiments are to explain just as examples, thereby, the present invention is not limited to be only these embodiments.

### Embodiment 1

### Classification and Identification of the Functional Ingredient of the Purified Natural Plant Vinegar

For the indentification of the functional ingredient of the purified natural plant vinegar, HP-INNOWAX(polyethyleneglycol crosslinked, 30 mm×0.25 mm(I.D.)×0.25 um(F.T.)) column and HP-5MS(5% phenylmethylsilicone crosslinked, 30 mm×0.25 mm(I.D.)×0.25 um(F.T.)) column were used, and HP 5890 Series II Plus GC and 5972 MSD as using instruments were utilized. In GC analysis, the oven was maintained at 50° C. for two minutes, and then increased 3° C. per 1 minute to 220° C., after that, maintained at 220° C. for five minutes. The injection port was set to 200° C., the detector was set to 250° C., the flow rate of helium was set at 0.72 ml/min and split ratio set at 10.

In GC-MSD analysis, the oven was maintained at 40° C. for five minutes, and then increased 3° C. per 1 minute to 220° C., after that, was maintained at 220° C. for five minutes. The flow rate of helium was set at 1 ml/min and split ratio set at 50. Accelerative voltage was set at 70 eV and the estimate and identification of most compounds was experimented compared with commercially available products or was used mass library data.

The purified natural plant vinegar was extracted with organic solvent, and then the ingredient of the extract was analyzed. 20 ml of the natural plant vinegar was put into 100 ml separatory funnel and was extracted with ether. Later, 5% NaHCO₃ was added to ether layer, and carbonyl fraction was separated from aqueous layer. After the separated aqueous layer was neutralized with 30% H₂SO₄, carbonyl fraction was obtained by extracting with ether. 2N NaOH was put into ether layer remained after extracting the carbonyl fraction, so phenolic fraction was separated from aqueous layer. After the separated aqueous layer was neutralized by same method when extracting the carbonyl fraction, phenolic fraction was obtained by extracting with ether. As above, neutral fraction and basic fraction were obtained from ether layer remained after the carbonyl fraction and the phenolic fraction were extracted. After the weight of the all ether layers was measured by vacuum concentration, contents about each fraction were calculated from that. As above, each extract was divided into acid fraction, phenolic fraction, neutral fraction and basic fraction by acid and alkali treatment and ingredients of each fraction was analyzed by using GC and GC-MS. The results are showed following in table 1.

TABLE 1

| | Fraction | | |
|---|---|---|---|
| Fraction | ratio(%) | Ingredient (compound) | rate(%) |
| Neutral | 24.8% | Piperonal | 1.61 |
| fraction | | Coumarin | 0.4 |
| | | 1-indanone | 2.02 |
| | | N.D.(no detected) | 95.97 |
| | | Total | 100 |
| Acid | 25.3% | acetic acid | 13.44 |
| fraction | | Maltol | 7.91 |
| | | Acetophenone | 1.57 |
| | | N.D. | 77.08 |
| | | Total | 100 |
| Phenolic | 47.0 | Guaiacol | 30.60 |
| fraction | | 4-methyl guaiacol | 0.60 |
| | | 4-ethyl guaiacol | 1.91 |
| | | 4-propyl guaiacol | 2.34 |
| | | Vanillin | 3.17 |
| | | 4-(2-propio)vanillone | 0.11 |
| | | 4-(1-propio)vanillone | 3.40 |
| | | Eugenol | 3.62 |
| | | E-isoeugenol | 0.21 |
| | | aceto-vanillone | 1.45 |
| | | Syringol | 28.40 |
| | | 4-methyl syringol | 4.47 |
| | | 4-ethyl syringol | 2.66 |
| | | 4-propyl syringol | 1.30 |
| | | Syringaldehyde | 1.49 |
| | | 4-(2-propio)-syringone | 0.60 |
| | | 4-(1-propio)-syringone | 0.23 |
| | | 4-(2-propenyl)-syringol | 0.32 |
| | | E-4-(1-propenyl)-syringol | 0.83 |
| | | Z-4-(1-propenyl)-syringol | 1.40 |
| | | Acetosyringone | 0.05 |
| | | N.D. | 10.84 |
| | | Total | 100 |
| Basic | 2.9 | 3-ethylphentane | 37.93 |
| fraction | | 4-methylene cyclohexanone | 17.24 |
| | | 2,6-di-tert-butylquinone | 27.59 |
| | | N.D. | 17.24 |
| | | Total | 100 |
| Total | 100 | | |

The rate (47%) of phenolic fraction was the highest and the rate (2.9%) of basic fraction was the least among classified fractions. In the phenolic fraction, the guaialcol compounds and the syringol compounda were main ingredients of phenolic fraction as the rate of 89.16%. The guaialcol compounds and the syringol compounds are produced by decomposed guaiacyl unit and syringyl unit of lignin, a constituent of timber by heat. These compounds are also sorts of phenolic acid compound known to conduct a strong antioxidant effect in vivo. The guaialcol compounds and the syringol compounds may be functional ingredients of the purified natural plant vinegar based on the results.

DPPH scavenging ability, as described later, was evaluated by using each fraction. As a result, the effect of phenolic fraction was confirmed to be much better than that of other fractions. The results are showed in table 2.

TABLE 2

| | Conc. (ul/ml) | | | | |
|---|---|---|---|---|---|
| | 10 | 50 | 100 | 250 | 500 |
| Acid fraciton | 15.49 ± 8.43 | 32.15 ± 6.94 | 45.29 ± 7.36 | 50.54 ± 6.29 | 56.50 ± 7.47 |
| Phenolic fraction | 64.70 ± 7.10 | 88.23 ± 6.53 | 90.23 ± 4.01 | 95.88 ± 2.68 | 98.41 ± 3.49 |
| Neutral fraction | 2.34 | 3.39 | 2.59 | 2.01 | 1.94 |
| Basic fraction | 1.96 ± 4.25 | 3.33 ± 4.61 | 8.43 ± 4.67 | 9.61 ± 6.59 | 12.25 ± 3.54 |

### Embodiment 2

### The Evaluation of the Antioxidant Effect

DPPH (1,1-diphenyl-2-picrylhydrazin) scavenging ability and antioxidant enzyme activity measurement of the pharmaceutical composition of the present invention were conducted in order that the pharmaceutical composition (phenolic fraction of embodiment 1) of the natural plant vinegar comprising the guaiacol compounds and the syringol compounds, extracted from the natural plant vinegar, is used as a natural antioxidant.

<DPPH Free Radical Scavenging Ability>

4 ml of samples with 0.1, 0.05, 0.005, 0.001, 0.0005 and 0.0001 mg/ml diluted with methanol and 1 ml of 0.1 mM DPPH methanol solution were put into test tubes and mixed well, and left in a dark place for 30 minutes. Then absorbance of the mixture at 520 nm was read and compared with that of the BHT standard solution. The reducing power of samples can be indicated as scavenging activity (SC50) and SC50 is the concentration of samples that makes the concentration of DPPH reduce to 50%. The results are showed in table 3.

TABLE 3

| | Free radical removal ability (%) | | | |
|---|---|---|---|---|
| | Conc. (weight %) | | | |
| | 0.1 | 1 | 5 | 10 |
| Composition | 14.07 ± 4.38 | 59.93 ± 3.22 | 89.40 ± 4.81 | 92.66 ± 5.12 |
| of the | | | | |
| present | | | | |
| invention | | | | |

| | Free radical removal ability (%) | | | |
|---|---|---|---|---|
| | Conc. (ug/ml) | | | |
| | 10 | 50 | 100 | 500 |
| BHT | 23.30 ± 1.94 | 68.41 ± 1.55 | 87.73 ± 0.64 | 96.03 ± 0.12 |

As shown in the result of table 3, the antioxidant effect of the composition of the present invention comprising the guaiacol compounds and the syringol compounds, extracted from the natural plant vinegar is excellent. The antioxidant ability of the 10% solution of the composition in the present invention is similar to that of 500 ug/ml of an artificial antioxidant agent, BHT.

<Antioxidant Enzyme Activity Measurement and Contents Measurement of MDA, AD and AH>

1 weight % liquid preparation of the composition in the present invention was administrated to mouse (SD) for two weeks, and bromobenzene (BB) was injected peritoneally at intervals of 12 hours for two days. 24 hours later from BB peritoneal injection, antioxidant mechanism was investigated by sacrificing mouse. The results are showed in table 4. The phenolic fraction of the embodiment 1 was used as the composition of the present invention.

TABLE 4

| Antioxidant | | BB | | Glutathion |
|---|---|---|---|---|
| enzyme | Test group | (mg/kg) | Dose (mg/kg) | S-trasnferase* |
| Glutathion | Normal group | — | — | 186.4 ± 17.7 |
| S-trasnferase | Control group | 460 | — | 143.8 ± 7.68 |
| | 1 weight % | 460 | 100 | 155.9 ± 8.61 |
| | composition | | | |
| Epoxide | Normal group | — | — | 14.80 ± 0.60 |
| Hydroxylase | Control group | 460 | — | 4.16 ± 0.13 |
| | 1 weight % | 460 | 100 | 9.37 ± 0.36 |
| | composition | | | |

*Conjugated 2,4-ninitrobenzene-glutathione nmole/mg protein/min

Glutathione S-transferase is antioxidant enzyme protecting tissues from damage by detoxification of glutathione radical produced. Epoxide hydroxylase is antioxidant enzyme catalyzing that high-reactive epoxide is hydrated into stable and low-reactive dihydrodiol product. As known as the results of table 4, 1 weight % liquid preparation of the composition of the present invention increased the activity of Glutathione S-transferase and epoxide hydroxylase in 8.41% and 125.1% respectively.

MDA (malondialdehyde) is a substance, which indicates lipid peroxides in total. The product increase of MDA means an increase of free radical like harmful oxygen and the damage of tissues increases by an increasing of MDA. Formaldehyde (AD) and P-aminophnal (AH) are metabolites having similar function as free radical generated from microsome of liver by material causing liver damage and they induce liver damage. Measured results of their contents are showed in table 5.

TABLE 5

| | BB | | | |
|---|---|---|---|---|
| Test group | (mg/kg) | Dose (mg/kg) | MDA of tissue(nmole/g) | |
| Normal group | — | — | 18.0 ± 1.18 | |
| Control group | 460 | — | 56.4 ± 1.77 | |
| 1 weight % | 460 | 100 | 41.2 ± 3.78 | |
| composition | | | | |
| | | | AD | AH |
| | BB | | nmole/mg | nmole/mg |
| Test group | (mg/kg) | Dose (mg/kg) | protein/min. | protein/min. |
| Normal group | — | — | 4.17 ± 0.24 | 0.64 ± 0.090 |
| Control group | 460 | — | 9.34 ± 0.37 | 1.26 ± 0.087 |
| 1 weight % | 60 | 100 | 7.88 ± 0.28 | 0.94 ± 0.073 |
| composition | | | | |

As shown in results of table 5, 1 weight % liquid preparation of the composition of the present invention decreased the content of MDA, AD and AH increased by BB, in 36.89%, 18.52% and 46.87% respectively. That means the composition of the present invention has the prominent antioxidant effect.

### Embodiment 3

### The Evaluation of Blood Glucose Level Control Effect

Improvement effect about blood glucose, blood total cholesterol, triglyceride and HbAlc was investigated. A C57BI/KsJ db/db mouse is known well as a tested animal of Type II diabetes. the present study also investigates improvement effect of Type I diabetes.

Thirty male mice of seven-weeks-old C57BI/KsJ-db/db were separated each 10 per a group, and the phenolic fraction of embodiment 1 and distilled water were administrated orally by using feeding needle at every 10-12 am for six weeks. Sample picking was conducted as blood picking and fat picking, and an intake investigation of water and feed and a weight investigation were conducted during breeding animals. Concentration of blood glucose was measured respectively at pre-feeding and post-feeding 30, 60, 90, 120 minutes when it was 2 weeks, 4 weeks and 6 weeks. After sacrificing tested animals, the total fatty weight of abdomen and epididymis was measured. The concentration of blood HbAlc was measured in tested animals for six weeks, and the blood was isolated from the heart, and the blood was analyzed at Seoul Clinical Laboratories. Concentration. measurement of blood tryglyceride and blood cholesterol was analyzed with analysis kit. All data were disposed statistically by using SAS package, and the results were showed as an average±the standard deviation. The concentration of blood glucose, blood triglyceride, total cholesterol and HbAlc between a control group and each test group was analyzed though t-test. The results are showed in table 6, 7, 8, 9, 10 and 11 respectively.

The weight of fat isolated from abdomen and epididymis is showed in table 6.

TABLE 6

| Sample | Weight of fat (g) |
|---|---|
| Control group | 12.4 ± 1.4 |
| The composition of the present invention 25 mg | 2.52 ± 0.2*** |
| The composition of the present invention 50 mg | 3.11 ± 10.4*** |
| The composition of the present invention 100 mg | 2.90 ± 0.2*** |

Significantly difference
*p < 0.05,
**p < 0.01,
***p < 0.001

As known in table 6, the fatty weight of all groups, to which the composition of the present invention was administrated for six weeks, was meaningfully less than that of control group (p<0.001). In particular, the group, to which 25 mg of the composition of the present invention was administrated, had the least fatty weight among all administrated groups.

Concentration changes of blood glucose depending on each time of orally administrating glucose for two weeks are showed in table 7.

TABLE 7

| | Pre-feeding | Post-feeding | Post-feeding | Post-feeding | Post-feeding |
|---|---|---|---|---|---|
| | 0 minute | 30 minutes | 60 minutes | 90 minutes | 120 minutes |
| Control group | 472.5 ± 10.9 | 600 ± 20.6 | 570.1 ± 8.6 | 560.2 ± 14.5 | 547.8 ± 9.2 |
| The composition | 292.2 ± 39.5 | 571 ± 31.1 | 570.7 ± 44.4 | 531.7 ± 57.5 | 478.3 ± 39.4 |
| of the present | | | | | |
| invention 25 mg | | | | | |
| The composition | 249.2 ± 33.9 | 527.1 ± 19.7 | 435.7 ± 19.7 | 348 ± 35.1 | 296.8 ± 33.4 |
| of the present | | | | | |
| invention 50 mg | | | | | |
| The composition | 192.3 ± 19.1 | 485.4 ± 13.5 | 398.9 ± 23.8 | 365.9 ± 20.3 | 291.3 ± 19.1 |
| of the present | | | | | |
| invention 100 mg | | | | | |
| Negative control | 112.7 ± 15.6 | 278 ± 10.4 | 177.5 ± 7.8 | 132.5 ± 12 | 130 ± 5.7 |
| group | | | | | |

As known in table 7, after 2 weeks from administrating 25 mg, 50 mg and 100 mg of the composition of the present invention, the glucose concentration of all test groups was less than that of the control group. In particular, the least concentration was showed 5 in the group administrating 50 mg and 100 mg of the composition of the present invention. In addition, a significant difference was showed in the group administrating 50 mg and 100 mg of the composition of the present invention (p<0.001).

Concentration changes of blood glucose depending on time of orally administrating glucose for four weeks are showed in table 8.

TABLE 8

| | Preprandial | Postcibal | Postcibal | Postcibal | Postcibal |
|---|---|---|---|---|---|
| | 0 minutes | 30 minutes | 60 minutes | 90 minutes | 120 minutes |
| Control group | 414 ± 12.5 | 555.3 ± 10.5 | 513.2 ± 18.6 | 487.6 ± 10.2 | 459.5 ± 20.1 |
| The composition | 309.4 ± 34.5 | 503.1 ± 79 | 456.1 ± 76.8 | 408.4 ± 81.7 | 337.9 ± 50.3 |
| of the present | | | | | |
| invention 25 mg | | | | | |
| The composition | 249.2 ± 33.9 | 527.1 ± 41.2 | 459 ± 47.8 | 326.5 ± 29.9 | 288.1 ± 30.4 |
| of the present | | | | | |
| invention 50 mg | | | | | |
| The composition | 216.7 ± 21.8 | 500.7 ± 17.8 | 398.1 ± 15.7 | 336.4 ± 19.5 | 307.3 ± 20.1 |
| of the present | | | | | |
| invention 100 mg | | | | | |
| Negative control | 104.7 ± 8.5 | 205.7 ± 9.2 | 140.3 ± 9 | 135.7 ± 28.3 | 124.7 ± 19.6 |
| group | | | | | |

As known in table 8, after 4 weeks from administrating 25 mg, 50 mg and 100 mg of the composition of the present invention, the glucose concentration of all test groups was less than that of the control group. The blood sugar of all test groups at 120 minutes was also measured similarly.

Concentration changes of blood glucose depending on time of orally administrating glucose for six weeks are showed in following table 8.

TABLE 9

| | Pre-feeding | Post-feeding | Post-feeding | Post-feeding | Post-feeding |
|---|---|---|---|---|---|
| | 0 minute | 30 minutes | 60 minutes | 90 minutes | 120 minutes |
| Control group | 511.8 ± 17.7 | 600 ± 12.3 | 578.6 ± 27.1 | 564.9 ± 15.7 | 524.7 ± 20.5 |
| The composition | 320.3 ± 30.3 | 582 ± 21.6 | 570.5 ± 48.9 | 480 ± 85.9 | 371.5 ± 40.5 |
| of the present | | | | | |
| invention 25 mg | | | | | |
| The composition | 352.8 ± 26.9 | 565.1 ± 43.5 | 451.4 ± 29.2 | 379.6 ± 6.1 | 377 ± 20.9 |
| of the present | | | | | |
| invention 50 mg | | | | | |
| The composition | 281.4 ± 14.2 | 530.5 ± 5.5 | 518.8 ± 30.5 | 393.8 ± 25 | 346 ± 20.1 |
| of the present | | | | | |
| invention 100 mg | | | | | |
| Negative control | 113.7 ± 5 | 230.5 ± 12.4 | 195.9 ± 10.2 | 176.4 ± 10.7 | 155.7 ± 25.2 |
| group | | | | | |

As known in table 9, after 4 weeks from administrating 25 mg, 50 mg and 100 mg of the composition of the present invention, the glucose concentration of all test groups was lower than that of the control group. In all administrated groups, the glucose concentration of pre-feeding and post-feeding 120 minutes was measured similarly. The glucose concentration on the 4th day after administration was similar to that on the 6^{th} day after administration.

The concentration of blood HbAlc is showed in table 10.

TABLE 10

| Sample | HbA1c (%) |
|---|---|
| Control group | 6.5 ± 0.6 |
| The composition of the present invention 25 mg | 6.2 ± 0.2 |
| The composition of the present invention 50 mg | 6.1 ± 1.0 |
| The composition of the present invention 100 mg | 6.2 ± 0.7 |

The concentration of blood HbAlc is one of important indexes for diabetic patients. The composition of the present invention was not meaningful statistically compared with the control group, but the HbAlc concentration of the composition was less than that of the control group.

The concentration of plasma neutral fat and total cholesterol is showed in table 11.

TABLE 11

| | Triglyceride (neutral | Total cholesterol |
|---|---|---|
| Sample | fat) (mg/dl) | (mg/dl) |
| Control group | 94.3 ± 27.1 | 124.2 ± 15.0 |
| The composition of the present | 61.0 ± 4.7 | 156.7 ± 19.4 |
| invention 25 mg | | |
| The composition of the present | 79.4 ± 4.4 | 125.9 ± 2.5 |
| invention 50 mg | | |
| The composition of the present | 70.0 ± 11.3 | 119.0 ± 6.5 |
| invention 100 mg | | |

Plasma neutral fat of the composition of the present invention was less than that of the control group, and that of 25 mg administration group of the composition of the present invention was the least and the significant difference was recognized (p<0.001). In groups administrating 50 mg and 100 mg of the composition of the present invention, the significant difference was also recognized (p<0.05). In case of total cholesterol, the concentration of the groups administrating 50 mg and 100 mg of the composition of the present invention were similar to that of the control group, but the concentration of the group administrating 25 mg of the composition of the present invention was greater than that of the control group. A significant difference was recognized in all administration groups.

The results of blood glucose control function evaluation test are as follows. Feed consumption of all tested animals wasn't showed any difference among groups and weight changes of tested animal was less than the control group. The total weight of abdomen and epididymis fat in the test group was less than that of the control group and the glucose concentration depending on administration after two weeks, four weeks and six weeks of test group was less than that of control group at pre-feeding and post-feeding 30 minutes, 60 minutes, 90 minutes and 120 minutes. Blood glycosylated hemoglobin of the test group was less than that of control group. Blood neutral fat of the test group was less than that of the control group and blood total cholesterol of the test group have relatively similar level compared with that of the control group. As a result, the composition of the present invention used for the present test can be confirmed to have effects regulating blood glucose control and decreasing on accumulating fat.

### Embodiment 4

### The Evaluation of the Improvement Effect in Blood Flow

<The Evaluation of Platelet Aggregation Degree Induced by Thrombin and Collagen>

The platelet aggregation degree induced by thrombin and collagen by using the composition (a test group 1) of the present invention comprising 15.5 weight % of the guaiacol compounds and 25 weight % of the syringol compounds, extracted from the natural plant vinegar, and the composition (a test group 2) of the present invention including 15.5 weight % of the guaiacol compound, 25 weight % of the syringol compound and 0.5 weight % of green tea leaves extract was evaluated on the basis of the total weight of the composition. Platelet induces excessive thrombogenesis through activation and cohesion at damaged vascular sites, thereby platelet plays an important role in many vascular diseases (SiMinno and silver, 1983).

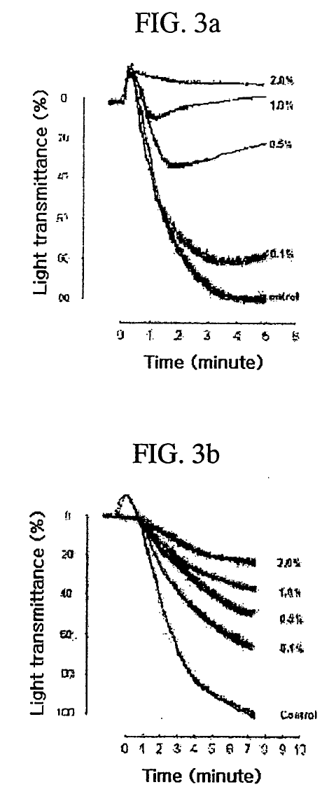

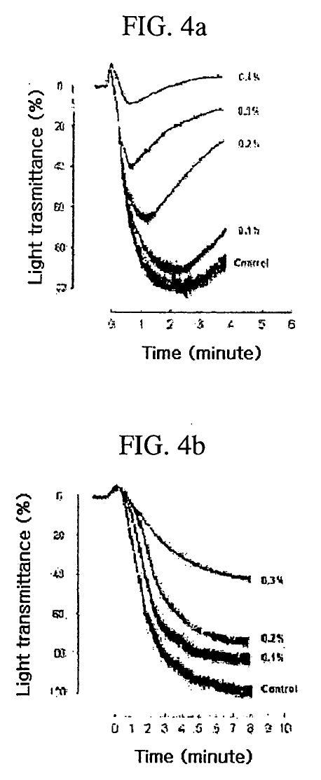

After preparing platelet, the test group 1 and the test group 2 were concentration-dependently cultured with the platelet in order to investigate effect of the test group 1 and the test group 2 on platelet. The test group 1 and the test group 2 were reacted with the platelet at 37° C. for 10 minutes. When the least unit or content of thrombin or collagen forming the maximal coagulation was added, the control group with water didn't have any change but the coagulation by thrombin and collagen was concentration-dependently inhibited in the test group 1 and the test group 2 reacted with platelet. The results are showed in FIGS. 3a, 3b, 4a and 4b. The coagulation degree of platelet was measured as turbidity change by using lumi-aggregometer. Light transmittance was 100% when all of the platelet was coagulated and light transmittance was 0% when platelet was not coagulated.

In these results, as for thrombin, IC50 was 0.386% in the test group 2 (N=3) and was 0.748% in the test group 1 (N=3). As for collagen, IC50 was 0.207% in the test group 2 (N=3) and was 0.547% in the test group 1 (N=3). As known in these results, the composition of the present invention could be confirmed to concentration-dependently inhibit platelet aggregation in evaluation of improvement in blood flow with thrombin and collagen. The test group 2 comprising the guaiacol compounds, the syringol compounds and green tea leaves extract was measured to have better effect than the test group 1.

<Assay of Serotonin Secretion>

The influence affected on the secretion of serotonin was evaluated depending on the composition of the present invention comprising 15.5 weight % of the guaiacol family compounds and 25 weight % of the syringol family compounds (the test group 1) and the composition of the present invention comprising 15.5 weight % of the guaiacol family compounds, 25 weight % of the syringol family compounds and 0.5 weight % of green tea leaves extract (the test group 2) and the composition comprising 0.5 weight % of green tea leaves extract (a comparative group 1).

0.1 U/mL of thrombin was added after the test group 1, the test group 2 and the comparative group 1 were cultured with platelet for 10 minutes. Then the amount of serotonin released for three minutes was assayed. Distilled water was used as the control group. The results are showed respectively in following table 12, 13 and 14.

TABLE 12

| | The secretion | | | |
|---|---|---|---|---|
| | of serotonin (%) | | | |
| Sample | 1^{st} time | 2^{nd} time | 3rd time | Average (%) |
| Control group | 65.65 | 61.80 | 52.62 | 60.02 |
| Test group 1-0.1% solution | 63.65 | 60.04 | 46.20 | 56.63 |
| Test group 1-0.5% solution | 59.11 | 56.61 | 37.84 | 51.19 |
| Test group 1-1% solution | 39.77 | 46.07 | 17.09 | 34.31 |
| Test group 1-2% solution | 2.85 | 17.53 | 10.19 | 10.19 |

TABLE 13

| | The secretion | | | |
|---|---|---|---|---|
| | of serotonin (%) | | | |
| Sample | 1^{st} time | 2^{nd} time | 3rd time | Average (%) |
| Control group | 56.23 | 53.18 | 63.94 | 57.78 |
| Test group 2-0.1% solution | 56.66 | 47.88 | 61.41 | 55.32 |
| Test group 2-0.5% solution | 58.24 | 36.61 | 47.79 | 47.55 |
| Test group 2-1% solution | 34.86 | 14.54 | 32.14 | 27.18 |
| Test group 2-2% solution | 10.93 | 3.67 | 11.00 | 8.53 |

TABLE 14

| | The secretion of | | | |
|---|---|---|---|---|
| | serotonin (%) | | | |
| | 1^{st} | 2^{nd} | 3rd | |
| Sample | time | time | time | Average (%) |
| Control group | 57.29 | 62.09 | 68.06 | 62.48 |
| comparative group 1-0.1% solution | 61.75 | 59.98 | 62.23 | 61.32 |
| comparative group 1-0.5% solution | 54.89 | 56.48 | 53.72 | 55.03 |
| comparative group 1-1% solution | 45.78 | 47.21 | 46.45 | 46.48 |
| comparative group 1-1.25% solution | 34.21 | 32.48 | 34.44 | 33.71 |
| comparative group 1-1.5% solution | 21.54 | 23.41 | 22.79 | 22.58 |
| comparative group 1-2% solution | 10.56 | 12.01 | 10.85 | 11.14 |

As known in the results of the table 12, the table 13 and the table 14, the composition of the present invention, the test group 1 and the test group 2, concentration-dependently inhibited the secretion of serotonin by blocking a stimulus of platelet by thrombin in the test results. That means the composition of the present invention is useful in inhibiting thrombogenesis. For the test group 1, IC50 was showed in concentration of about 1% but for the test group 2, IC50 was showed in concentration of about 0.5% and for the comparative group 1, IC50 was showed in concentration of about 1.25%. That means the composition of the present invention comprising the guaiacol family compounds, the syringol family compounds and green tea leaves extract has more preferable effect and when green tea leaves extract is added to the composition of the present invention comprising the guaiacol compounds and the syringol compounds, the composition has synergy effect.

<Assay of Vasoconstriction Inhibition Effect by Phenylephrine>

Vasoconstriction inhibition effect by phenylephrine was assayed in order to evaluate vasoconstriction effect of the composition of the present invention comprising 15.5 weight % of the guaiacol family compound and 25 weight % of the syringol family compounds (the test group 1) and the composition of the present invention comprising 15.5 weight % of the guaiacol compounds, 25 weight % of the syringol compounds and 0.5 weight % of green tea leaves extract (the test group 2) and the composition comprising 0.5 weight % of green tea leaves extract (the comparative group 1).

After the thoracic aorta of white rat was pre-treated with 0.5 to 2% of test group 1 solution, 0.1 to 0.4% of the test group 2 solution and control group with water, phenylephrine was added to the test groups and control group from low concentration to high concentration. The results are showed in table 15, table 16 and table 17, respectively.

TABLE 15

| | | Contraction | | | |
|---|---|---|---|---|---|
| | Dose | (%, 90 mM K+) | | | |
| Sample | −log[PE(M)] | 1^{st} time | 2^{nd} time | 3^{rd} time | Average |
| Control group | 9 | 1.34 | −0.78 | 1.05 | 0.54 |
| | 8.5 | 0.89 | −1.75 | −0.70 | −0.52 |
| | 8.0 | 1.12 | −0.39 | 4.56 | 1.76 |
| | 7.5 | 2.68 | 6.04 | 18.25 | 8.99 |
| | 7.0 | 26.30 | 31.38 | 48.42 | 35.37 |
| | 6.5 | 51.60 | 51.85 | 65.26 | 56.24 |
| | 6.0 | 65.40 | 64.32 | 77.19 | 68.67 |
| | 5.5 | 76.10 | 77.19 | 82.80 | 78.70 |
| | 5.0 | 80.40 | 84.02 | 88.77 | 84.40 |
| Test group | 9 | −0.47 | −1.53 | 1.69 | −0.10 |
| 1-0.5% solution | 8.5 | −3.04 | 2.68 | 1.69 | 0.44 |
| | 8.0 | 0.23 | 4.02 | 4.22 | 1.49 |
| | 7.5 | 1.41 | 9.39 | 11.81 | 7.54 |
| | 7.0 | 21.50 | 40.23 | 47.68 | 36.47 |
| | 6.5 | 46.40 | 56.70 | 66.67 | 56.59 |
| | 6.0 | 62.50 | 71.84 | 79.75 | 71.36 |
| | 5.5 | 70.70 | 83.33 | 90.30 | 81.44 |
| | 5.0 | 74.50 | 89.46 | 98.31 | 87.42 |
| Test group | 9 | 1.61 | −0.50 | −1.16 | −0.02 |
| 1-2% solution | 8.5 | 0.00 | −0.74 | 0.87 | 0.04 |
| | 8.0 | 1.34 | 0.00 | 2.31 | 1.22 |
| | 7.5 | 2.15 | 3.71 | 9.82 | 5.23 |
| | 7.0 | 16.40 | 21.78 | 34.68 | 24.29 |
| | 6.5 | 44.40 | 48.02 | 60.98 | 51.13 |
| | 6.0 | 59.40 | 64.85 | 73.70 | 65.98 |
| | 5.5 | 72.60 | 80.20 | 84.39 | 79.06 |
| | 5.0 | 77.70 | 88.86 | 91.90 | 86.15 |

TABLE 16

| | | Contraction | | | |
|---|---|---|---|---|---|
| | Dose | (%, 90 mM K+) | | | |
| Sample | −log[PE(M)] | 1^{st} time | 2^{nd} time | 3^{rd} time | Average |
| Control group | 9 | 0.90 | −0.76 | 0.30 | 0.15 |
| | 8.5 | 3.90 | 0.50 | 0.00 | 1.47 |
| | 8.0 | 6.90 | 0.75 | 1.20 | 2.95 |
| | 7.5 | 23.72 | 19.40 | 15.87 | 19.66 |
| | 7.0 | 51.95 | 52.90 | 49.40 | 51.42 |
| | 6.5 | 67.27 | 67.00 | 61.68 | 65.32 |
| | 6.0 | 77.78 | 86.81 | 80.79 | 81.79 |
| | 55. | 84.38 | 90.43 | 85.82 | 86.88 |
| | 5.0 | 89.19 | 92.95 | 90.90 | 91.01 |
| Test group | 9 | −0.45 | −0.59 | 1.57 | 0.18 |
| 2-0.1% solution | 8.5 | −0.45 | 0.29 | 2.83 | 0.89 |
| | 8.0 | 0.00 | 0.30 | 2.89 | 1.06 |
| | 7.5 | 4.23 | 2.37 | 5.03 | 3.88 |
| | 7.0 | 18.26 | 18.34 | 16.35 | 17.65 |
| | 6.5 | 35.63 | 23.96 | 33.33 | 30.97 |
| | 6.0 | 44.54 | 39.65 | 45.91 | 43.37 |
| | 5.5 | 54.12 | 44.38 | 54.72 | 51.07 |
| | 5.0 | 58.13 | 48.22 | 55.97 | 54.11 |
| Test group 2-0.2% | 9 | 0.91 | −0.31 | 0.00 | 0.20 |
| solution | 8.5 | 0.23 | 0.61 | 0.55 | 0.46 |
| | 8.0 | 0.68 | 0.61 | 0.82 | 0.70 |
| | 7.5 | 0.23 | 2.14 | 1.10 | 1.16 |
| | 7.0 | 0.23 | 3.98 | 4.93 | 3.05 |
| | 6.5 | 8.9 | 22.94 | 18.63 | 16.82 |
| | 6.0 | 29.00 | 34.86 | 29.31 | 31.06 |
| | 5.5 | 34.58 | 40.57 | 39.45 | 38.20 |
| | 5.0 | 38.81 | 44.95 | 45.21 | 42.99 |
| Test group 2-0.4% | 9 | −1.39 | −1.02 | 1.26 | −0.38 |
| solution | 8.5 | −1.66 | 0.00 | 2.09 | 0.14 |
| | 8.0 | −1.19 | 2.03 | 2.93 | 1.26 |
| | 7.5 | 0.28 | 2.37 | 3.77 | 2.14 |
| | 7.0 | 3.40 | 7.46 | 3.77 | 4.88 |
| | 6.5 | 20.78 | 17.29 | 16.66 | 18.24 |
| | 6.0 | 27.42 | 29.83 | 22.55 | 26.60 |
| | 5.5 | 29.36 | 34.57 | 27.99 | 30.64 |
| | 5.0 | 30.75 | 41.02 | 28.41 | 33.39 |

TABLE 17

| | | Contraction | | | |
|---|---|---|---|---|---|
| | Dose | (%, 90 mM K+) | | | |
| Sample | −log[PE(M)] | 1^{st} time | 2^{nd} time | 3^{rd} time | Average |
| Control group | 9 | 0.35 | 0.24 | 0.60 | 0.40 |
| | 8.5 | 1.22 | 1.76 | 2.01 | 1.66 |
| | 8.0 | 1.98 | 2.24 | 3.98 | 2.73 |
| | 7.5 | 2.58 | 6.87 | 17.55 | 9.00 |
| | 7.0 | 24.38 | 30.78 | 40.47 | 31.88 |
| | 6.5 | 49.78 | 53.68 | 67.89 | 57.12 |
| | 6.0 | 60.88 | 67.42 | 79.05 | 69.12 |
| | 5.5 | 70.20 | 79.55 | 85.21 | 78.32 |
| | 5.0 | 80.96 | 85.04 | 88.99 | 85.00 |
| Comparative group | 9 | 0.11 | 0.44 | 0.55 | 0.37 |
| 1-0.5% solution | 8.5 | 0.89 | 2.45 | 3.65 | 2.33 |
| | 8.0 | 2.99 | 3.56 | 7.89 | 4.81 |
| | 7.5 | 8.57 | 10.02 | 15.55 | 11.38 |
| | 7.0 | 27.88 | 39.89 | 45.02 | 37.60 |
| | 6.5 | 69.45 | 70.02 | 65.78 | 68.42 |
| | 6.0 | 75.45 | 75.00 | 78.81 | 76.42 |
| | 5.5 | 78.98 | 80.21 | 79.95 | 79.71 |
| | 5.0 | 90.01 | 85.49 | 84.02 | 86.51 |
| Comparative group | 9 | 0.39 | 0.76 | 0.33 | 0.49 |
| 1-2% solution | 8.5 | 3.98 | 0.99 | 5.61 | 3.53 |
| | 8.0 | 8.02 | 5.98 | 14.44 | 9.48 |
| | 7.5 | 11.11 | 9.99 | 20.56 | 13.89 |
| | 7.0 | 32.48 | 29.54 | 35.23 | 32.42 |
| | 6.5 | 71.04 | 69.78 | 63.33 | 68.05 |
| | 6.0 | 70.44 | 72.41 | 75.82 | 72.89 |
| | 5.5 | 79.85 | 75.59 | 78.88 | 78.11 |
| | 5.0 | 84.69 | 86.53 | 88.87 | 86.70 |

PE means phenylephrine in above the table 15, the table 16 and the table 17. The test group 1 and the comparative group 1 didn't affect the vasoconstriction (see the table 15 and the table 17). On the while, the test group 2 comprising the guaiacol compounds, the syringol compounds and green tea leaves extract concentration-dependently decreased the contraction induced by phenylephrine (see the table 16). That means the composition of the present invention comprising the guaiacol compounds, the syringol compounds and green tea leaves extract has more preferable effect on improvement in blood flow than the test group 1 and the comparative group 1

The composition of the present invention comprising the guaiacol compounds and the syringol compounds, extracted from the natural plant vinegar, and the composition of the present invention comprising the guaiacol compounds, the syringol compounds and green tea leaves extract were confirmed to have inhibitory activity of platelet aggregation and inhibitory effect of vasoconstriction. That means the composition of the present invention can be used for improvement in blood flow in order to improve blood circulation.

### Embodiment 5

### The Evaluation of Treating Hangover by the Composition of the Present Invention

Ethanol and acetaldehyde concentration which are two toxic materials generated by drinking alcohol was assayed time-scale compared with control group. Water is employed as control group. The composition of the present invention comprising 15.5 weight % of the guaiacol family compound and 25 weight % of the syringol family compounds (the test group 1) and the composition of the present invention comprising 15.5 weight % of the guaiacol compounds, 25 weight % of the syringol compounds and 0.5 weight % of green tea leaves extract (the test group 2) and the composition comprising 0.5 weight % of green tea leaves extract (the comparative group 1).

<The Measurement of a Concentration Change of Blood Ethanol>

The concentration change of blood ethanol was showed in the table 8. The concentration change of blood ethanol was measured by following method. After test animals were fasted for 18 hours, test samples prepared with a solution having a suitable concentration. were orally administered Alcohol was orally administrated after 30 minutes, and blood was collected from orbit at 1, 3 and 5 hours after the administration and from the heart at 7 hours later after administration. The amount of ethanol in serum was measured by using ethanol measurement kit (Ethanol, Roche, Swizerland) after serum was isolated from blood by the centrifugation at 300 rpm for 15 minutes.

TABLE 18

| | Administration | Administration | Administration | Administration | AUC of |
|---|---|---|---|---|---|
| Sample | 1 hour later | 3 hours later | 5 hours later | 7 hours later | time-concentration |
| Control | 196.7 ± 25.2 | 180.7 ± 5.1 | 142.6 ± 26.1 | 99.3 ± 21.0 | 949.33 ± 108.74 |
| group | | | | | |
| Test group 1 | 47.2 ± 21.0 | 67.4 ± 4.2 | 39.1 ± 11.3 | 57.1 ± 31.9 | 398.65 ± 5.02 |
| | (p < 0.05) | (p < 0.001) | (p < 0.05) | (p < 0.05) | (p < 0.05) |
| Test group 2 | 144.7 ± 26.3 | 136.7 ± 20.8 | 100.0 ± 26.5 | 27.3 ± 13.0 | 655.00 ± 126.19 |
| | (p < 0.05) | (p < 0.05 | | (p < 0.05) | (p < 0.05) |
| Comparative | 187.7 ± 28.3 | 170.23 ± 18.8 | 130.40 ± 10.5 | 78.3 ± 23.0 | 876.68 ± 98.89 |
| group 1 | (p < 0.05) | (p < 0.05) | | (p < 0.05) | (p < 0.05) |

As known in the table 18, the highest concentration of blood alcohol was reached at 3 hours after administrating alcohol in the test group 1, and the blood alcohol concentration in the test group was lower at all time rather than the control group. When the concentration of blood alcohol in the control group was fixed as 100%, that of the test group 1 was reduced to 76% at 1 hour after administrating than the concentration of the blood alcohol of the control group. That of the test group 1 decreased respectively 63%, 73% and 43% at 3 hours, 5 hours and 7 hours. Significant changes showed in all time ranges.

Moreover, As the area under the curve (AUC) of time-concentration of blood ethanol of the control group, the test group 1, the test group 2 and the comparative group were compared, the area under the curve (AUC) of time-concentration of blood ethanol in the test group 1 was low significantly (p<0.05) compared with that of the control group reducing 58%. In these results, the composition of the present invention comprising the guaiacol compounds and the syringol compounds may reduce the concentration of blood alcohol elevated after administrating alcohol.

<The Measurement of a Concentration Change of Blood Acetaldehyde>

The measured results are showed in table 19. Concentration of blood acetaldehyde was measured by following method. After test animals were fasted for 18 hours, test substances prepared with a solution having a suitable concentration were orally administered. Alcohol was orally administrated after 30 minutes, and blood was collected from orbit at 1, 3 and 5 hours after the administration and from the heart at 7 hours after administration. The acetaldehyde concentration in serum was measured by using acetaldehyde measurement kit (Ethanol, Roche, Swizerland) after serum was isolated from blood by centrifugation at 3000 rpm for 20 minutes.

TABLE 19

| | Administration | Administration | Administration | Administration | AUC of |
|---|---|---|---|---|---|
| Sample | 1 hour later | 3 hours later | 5 hours later | 7 hours later | time-concentration |
| Control group | 0.30 ± 0.13 | 0.30 ± 0.09 | 0.39 ± 0.10 | 0.37 ± 0.08 | 2.05 ± 0.58 |
| Test group 1 | 0.39 ± 0.09 | 0.27 ± 0.01 | 0.24 ± 0.06 | 0.15 ± 0.01 | 1.57 ± 0.24 |
| | | | | (p < 0.05) | |
| Test group 2 | 0.17 ± 0.03 | 0.18 ± 0.07 | 0.11 ± 0.04 | 0.10 ± 0.05 | 0.86 ± 0.29 |
| | | | | (p < 0.05) | (p < 0.05) |
| Comparative | 0.26 ± 0.01 | 0.23 ± 0.05 | 0.24 ± 0.03 | 0.17 ± 0.02 | 1.85 ± 0.29 |
| group 1 | | | | (p < 0.05) | |

As known in the table 19, concentration of blood acetaldehyde was 0.37±0.08 mg % at 7 hours after administrating alcohol in the control group and was 0.15±0.01 mg % at 7 hours after administrating alcohol in the test group 1. therefore concentration in the test group 1 (p<0.05) was lower significantly than both the control group and the comparative group 1. Concentration of blood acetaldehyde was 0.10±0.05 mg % in at 7 hours from administrating alcohol in the test group 2, thereby, concentration of the test group 2 (p<0.05) was the lowest significantly between the test group 1 and the comparative group 1. The control group was 2.05±0.58, the test group 1 was 1.57±0.24, the test group 2 was 0.86±0.29 (p<0.05) and the comparative group 1 was 1.85±10.29 in AUC of blood acetaldehyde, thereby, the test group 2 was the lowest in the same case of concentration of blood acetaldehyde. These results mean the composition of the present invention comprising the guaiacol compounds and the syringol compounds is effective on treating hangover and the composition of the present invention which green tea leaves extract is added, is more preferable in treating hangover with a synergistic effect.

### Embodiment 6

### The Evaluation of Treating Atopic Dermatitis

The composition of the present invention was evaluated compared with Tacrolimus ointment (Protopics™, Fujisawa Korea Limited) commonly used for treating atopic dermatitis in order to evaluate the effect of treating atopic dermatitis by the composition of the present invention. The composition comprising 8.95 weight % of the guaiacol family compounds, 18.53 weight % of the syringol family compounds, 22.92 weight % of herbal extract comprising liquordice root (Glicyrrhizae Radix), Korean angelica (Angelicagigas), paeonia japonica, hoelen (Poria cocos Wolf), scutellaria root (Scutellaria baicalensis Georgi), schizandra fruit (Schizandra chinensis Baillon), ginger (Zingiber officinale) and cnidium rhizome (Cnidium officinale Makino) and 49.60 weight % of purified water was used as the composition of the present invention. Commercially available Tacrolimus ointment was used for treating atopic dermatitis as a positive control group and excipient, distilled water, was used as a negative control group.

NC/Nga mouse used as common animal model was used in order to evaluate the therapeutic agent of atopic dermatitis. NC/Nga mouse is an animal examined lesion similar to atopic dermatitis of human is generated after 6-7 weeks and lesion of asteatosis, wound which a scab forms over and small crystal on about 16-18 weeks, if NC/Nga mouse grows up exposing to general environment. 1000 mg/kg body weight of the negative control group, the positive control group and the composition of the present invention were diluted with distilled water, and then the diluted samples were orally administrated once a day for 4 weeks. 4 weeks later, the skin was partially dissected, and then evaluated for its damage degree.

Gross autopsy was conducted for total evaluation. Any particular abnormality wasn't examined as finding of internal organ. However, 7 rats in the negative control group, 4 rats in the positive control group and 2 rats in the composition of the present invention were grossly examined as animals with small wound over dorsal below skin, in external finding of skin. That shows a significant difference.

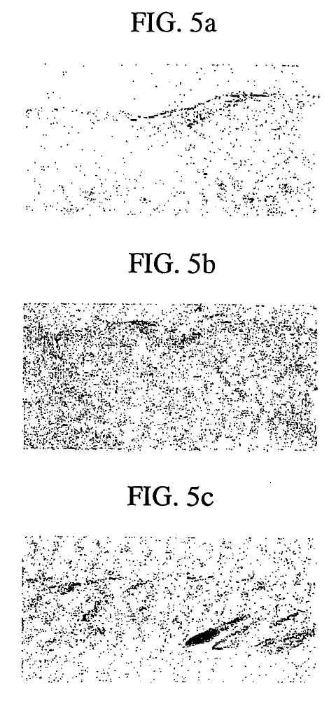

Microscopic assay was performed for histopathological examination. In results, the skin lesion was evaluated as skin ulcer, infiltration of acute-chronic inflammatory cell, stratified thickness of skin epithelial cell and scar. Each result is showed in table 20, 21, 22, 23 and FIGS. 5a, 5b, 5c.

TABLE 20
Skin ulcer (Damage degrec)

| | Number of | No | Weak | Middle | Strong |
|---|---|---|---|---|---|
| Sample | test group | damage | damage | damage | damage |
| Negative control group | 8 | 4 | 1 | 3 | 0 |
| Positive control group | 8 | 4 | 4 | 0 | 0 |
| The composition of the | 7 | 6 | 1 | 0 | 0 |
| present invention | | | | | |

TABLE 21
Infiltration of inflammatory cell (Damage degree)

| | Number of | No | Very weak | Weak | Middle |
|---|---|---|---|---|---|
| Sample | test group | damage | damage | damage | damage |
| Negative control | 8 | 0 | 3 | 3 | 2 |
| group | | | | | |
| Positive control | 8 | 0 | 4 | 3 | 1 |
| group | | | | | |
| The composition of | 7 | 3 | 3 | 1 | 0 |
| the present | | | | | |
| invention | | | | | |

TABLE 22
Thickening (Damage degree)

| | Number of | No | Very weak | Weak | Middle |
|---|---|---|---|---|---|
| Sample | test group | damage | damage | damage | damage |
| Negative control | 8 | 0 | 2 | 5 | 1 |
| group | | | | | |
| Positive control | 8 | 0 | 3 | 3 | 2 |
| group | | | | | |
| The composition of | 7 | 4 | 2 | 1 | 0 |
| the present | | | | | |
| invention | | | | | |

TABLE 23
Scar (The number)

| | Number of test | | | | | | |
|---|---|---|---|---|---|---|---|
| Sample | group | 0 | 1 | 2 | 3 | 4 | 5 |
| Negative control group | 8 | 1 | 2 | 1 | 2 | 1 | 1 |
| Positive control group | 8 | 4 | 3 | 1 | 0 | 0 | 0 |
| The composition of the | 7 | 5 | 1 | 1 | 0 | 0 | 0 |
| present invention | | | | | | | |

In 3 cm skin in length, the negative control group showed 50% occurrence of the skin ulcer, but the group of the composition of the present invention showed 14.3% occurrence of skin ulcer. Inflammation was examined in hypoderm and around ulcer and all negative control group showed the infiltration of acute or chronic inflammatory cell but the composition group of the present invention showed only about 57% of inflammatory finding. All of negative control group showed the partial thickening of the epithelial cell. Meanwhile, about 57% among the group treated with the composition of the present invention showed normal condition. The scar was examined (87.5%) in 7 rats among 8 rats of the negative control group, however, was examined (28.6%) only 2 rats among 7 rats of the composition group of the present invention. The group treated with composition of the present invention showed better results than the positive control group in both gross finding and histopathological examination. These results show that the composition of the present invention has excellent effect on treating atopic dermatitis.

### INDUSTRIAL APPLICABILITY

The present invention provides the pharmaceutical composition comprising the guaiacol family compounds and the syringol family compounds extracted from natural plant vinegar. The pharmaceutical composition of the present invention has a effect on treating oxidative toxicity, regulating blood glucose level, improving blood flow, treating hangover and treating atopic dermatitis as well as the safe composition to be free from acute toxicity, genetic toxicity, subacute toxicity etc. The pharmaceutical composition of the present invention can be used as an agent or an ingredient of health functional food.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

1. Pharmaceutical composition comprising the guaiacol family compounds shown by the following formula 1 and the syringol family compounds shown by the following formula 2, extracted from the natural plant vinegar, 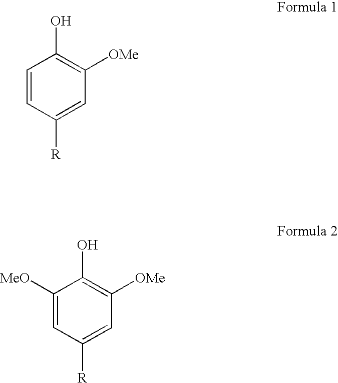
where, in the formulas 1 and 2, R is hydrogen, alkyl, oxoalkyl or alkenyl.

2. Pharmaceutical composition according to claim 1,
wherein contents of the guaiacol family compounds and the syringol family compounds are respectively 10⁻⁶ to 90 weight % and 10⁻⁶ to 90 weight % by weight based on the total weight of the compound.

3. Pharmaceutical composition for treating oxidative toxicity comprising the guaiacol family compound shown by the formula 1 of claim 1 and the syringol family compounds shown by the formula 2 of claim 1, extracted from natural plant vinegar.

4. Pharmaceutical composition according to claim 3,
wherein pharmaceutical composition is used to prevent or treat stroke, parkinson's disease, heart disease, ischemia, arteriosclerosis, dermatological disease, digestive disorder, inflammation, rheumatism, autoimmune disease or aging.

5. Pharmaceutical composition for regulating blood glucose level comprising the guaiacol family compounds shown by the formula 1 of claim 1 and the syringol family compounds shown by the formula 2 of claim 1, extracted from natural plant vinegar.

6. Pharmaceutical composition for improving blood flow comprising the guaiacol family compounds shown by the formula 1 of claim 1 and the syringol family compounds shown by the formula 2 of claim 1, extracted from natural plant vinegar.

7. Pharmaceutical composition for treating hangover comprising the guaiacol family compounds shown by the formula 1 of claim 1 and the syringol family compounds shown by the formula 2 of claim 1, extracted from natural plant vinegar.

8. Pharmaceutical composition according to claim 6,
wherein the composition further comprises a green tea leaves extract.

9. Pharmaceutical composition according to claim 8,
wherein contents of the guaiacol compound, the syringol compound and green tea leaves extract are 10⁻⁶ to 90 weight %, 10⁻⁶ to 90 weight % and 0.01 to 30 weight % respectively by weight based on the total weight of the composition.

10. Pharmaceutical composition for treating atopic dermatitis comprising the guaiacol family compounds shown by the formula 1 of claim 1 and the syringol family compounds shown by the formula 2 of claim 1, extracted from natural plant vinegar.

11. Pharmaceutical composition according to claim 10,
wherein the composition further comprises more than one herbal extract selected from a group consisted of Korean angelica (Angelicagigas) extract, cnidium rhizome (Cnidium officinale Makino) extract, liquordice root (Glicyrrhizae Radix) extract, hoelen (Poria cocos Wolf) extract, scutellaria root (Scutellaria baicalensis Georgi) extract, paeonia japonica extract, schizandra fruit (Schizandra chinensis Baillon) extract and ginger extract.
